(19) United States
(12) Patent Application Publication
(10) Pub. No.: US 2024/0254463 A1
(43) Pub. Date: Aug. 1, 2024

(54) ENGINEERED STABLE NUCLEIC ACID-GUIDED NUCLEASES

(71) Applicant: VedaBio, Inc., San Diego, CA (US)

(72) Inventor: Anurup Ganguli, San Diego, CA (US)

(21) Appl. No.: 18/424,815

(22) Filed: Jan. 28, 2024

Related U.S. Application Data
(60) Provisional application No. 63/442,737, filed on Feb. 1, 2023.

(51) Int. Cl. C12Q 1/6818; C12N 9/22; C12N 15/11; C12Q 1/6806

(52) U.S. Cl. CPC C12N 9/22; C12N 15/11; C12N 2310/20; C12Q 1/6806; C12Q 1/6818

(57) ABSTRACT

The present disclosure relates to stabilization variant engineered nucleic acid-guided nucleases that are used in CRISPR-based cascade assay systems to detect one or more target nucleic acids in a sample. The cascade assay systems provide signal boost upon detection of target nucleic acids without boost of the target nucleic acids. The stabilization variant engineered nucleic acid-guided nucleases are particularly useful in point-of-care applications, in the field, and as components of assay kits.

### RELATED APPLICATIONS

This application claims priority to U.S. Ser. No. 63/442,737, filed 1 Feb. 2023, which is incorporated by reference in its entirety.

### INCORPORATION BY REFERENCE OF SEQUENCE LISTING

Submitted herewith is an electronically filed sequence listing via EFS-Web a Sequence Listing XML, entitled “VB011US1_seqlist_20240116”, created 16 Jan. 2024, which is 25.5 kb in size. The sequence listing is part of the specification of this specification and is incorporated by reference in its entirety.

### FIELD OF THE INVENTION

The present disclosure relates to a variant engineered nucleic acid-guided nucleases that are used in CRISPR-based cascade assay systems to detect one or more target nucleic acids in a sample. The cascade assay systems provide signal boost upon detection of target nucleic acids without amplification of the target nucleic acids.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Rapid and accurate identification of, e.g., infectious agents, microbe contamination, variant nucleic acid sequences that indicate the presence of diseases such as cancer or contamination by heterologous sources is important in order to select correct treatment; identify tainted food, pharmaceuticals, cosmetics and other commercial goods; and to monitor the environment including identification of biothreats. Classic PCR and nucleic acid-guided nuclease or CRISPR (clustered regularly interspaced short palindromic repeats) detection methods rely on pre-amplification of target nucleic acids of interest to enhance detection sensitivity. However, amplification increases time to detection and may cause changes to the relative proportion of nucleic acids in samples that, in turn, lead to artifacts or inaccurate results. Improved technologies that allow very rapid and accurate detection of nucleic acids are therefore needed for timely diagnosis and treatment of disease, to identify toxins in consumables and the environment, as well as in other applications.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to variant engineered nucleic acid-guided nucleases that may be used in cascade assay systems that allow for quick target nucleic acid detection from very small samples without the need for amplification of the target nucleic acid(s). The variant nucleic acid-guided nucleases have been engineered to increase the stability of and decrease the aggregation properties of the wildtype LbCas12a (Lachnospriaceae bacterium Cas12a) nuclease (i.e., the “stabilization variant nuclease”) by preventing disulfide bridge formation within and between the stabilization variant nucleases.

Thus, there is provided in one embodiment, stabilization variant LbCas12a nucleases having sequences comprising SEQ ID NOs: 2-12; comprising C10S (SEQ ID NO: 2); C175S (SEQ ID NO: 3); C565S (SEQ ID NO: 4); C632S (SEQ ID NO: 5); C805S (SEQ ID NO: 6); C912S (SEQ ID NO: 7); C965S (SEQ ID NO: 8); C1090S (SEQ ID NO: 9); C1116S (SEQ ID NO: 10); C965S/C1090S (SEQ ID NO: 11); and C565S/C805S/C912S/C965S/C1090S/C1116S (SEQ ID NO: 12), as well as other combinations of these variants. Also provided are stabilization variant LbCas12a nuclease molecules that aggregate with each other less than wildtype LbCas12a nuclease molecules.

In other embodiments there is provided a reaction mixture comprising: a first ribonucleoprotein (RNP) complex (RNP1) comprising a first nucleic acid-guided nuclease and a first guide RNA (gRNA); wherein the first gRNA comprises a sequence complementary to a target nucleic acid of interest, and wherein the first nucleic acid-guided nuclease exhibits both cis-cleavage activity and trans-cleavage activity; a second ribonucleoprotein complex (RNP2) comprising a second nucleic acid-guided nuclease and a second gRNA that is not complementary to the target nucleic acid of interest; wherein the second nucleic acid-guided nuclease exhibits both cis-cleavage activity and trans-cleavage activity; and a plurality of blocked nucleic acid molecules comprising a sequence complementary to the second gRNA, wherein the blocked nucleic acid molecules cannot activate the RNP1 or the RNP2, and wherein at least one of the first and second nucleic acid-guided nucleases is the stabilization variant LbCas12a nuclease of claim 1.

In some aspects of this embodiment, the plurality of blocked nucleic acid molecules comprises: a first region, which is the sequence complementary to the second gRNA; one or more second regions not complementary to the first region; and one or more third regions complementary and hybridized to the first region, wherein cleavage of the one or more second regions results in dehybridization of the third region from the first region, resulting in an unblocked nucleic acid molecule.

In some aspects, a K_{d} of the blocked nucleic acid molecule binding to the RNP2 complex is at least 10¹⁰-fold greater than a K_{d} of the blocked nucleic acid molecule binding to the RNP2 when unblocked, and in some aspects, a K_{d} of the blocked nucleic acid molecule binding to the RNP2 complex is at least 10⁹-fold greater, at least 10⁸-fold greater, at least 10⁷-fold greater, at least 10⁶-fold greater, or at least 10⁵-fold greater than a K_{d} of the blocked nucleic acid molecule binding to the RNP2 when unblocked.

In some aspects, the reaction mixture comprises reporter moieties, and in some aspects, the reporter moiety comprises a DNA, RNA or chimeric nucleic acid molecule and/or a fluorescent, chemiluminescent, radioactive, colorimetric or other optical signal.

Other embodiments provide a method of detecting a target nucleic acid of interest in a sample comprising the steps of: providing the reaction mixture described above; contacting the reaction mixture with the sample under conditions that allow the target nucleic acid of interest in the sample to bind to RNP1; wherein upon binding of the target nucleic acid of interest RNP1 becomes active initiating trans-cleavage of at least one of the blocked nucleic acid molecules thereby producing at least one unblocked nucleic acid molecule, and wherein the at least one unblocked nucleic acid molecule binds to RNP2 initiating cleavage of at least one further linear blocked nucleic acid molecule; and detecting the cleavage products, thereby detecting the target nucleic acid of interest in the sample.

In some aspects, the RNP2 in the reaction mixture comprises one of the stabilization variant LbCas12a nucleases comprising SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 is a simplified diagram of a CRIPSR cascade reaction. In brief, a target nucleic acid binds to a first preassembled ribonucleoprotein (RNP1) complex and activates both cis- and trans-endonuclease activity, which in turn triggers activation of the trans-endonuclease activity of second preassembled ribonucleoprotein (RNP2) complexes. Each newly activated RNP2 complex activates more RNP2 complexes, with consequent cleavage of reporter moieties, which generate an exponential signal change in response to the exponential activation of RNP2 complexes.

FIG. 2 is a diagram showing in detail the sequence of steps in an exemplary cascade assay utilizing blocked nucleic acid molecules.

It should be understood that the drawings are not necessarily to scale, and that like reference numbers refer to like features.

### Definitions

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

All of the functionalities described in connection with one embodiment of the compositions and/or methods described herein are intended to be applicable to the additional embodiments of the compositions and/or methods except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

Note that as used herein and in the appended claims, the singular forms “a,” “an,” and “the” include plural referents unless the context clearly dictates otherwise. Thus, for example, reference to “a cell” refers to one or more cells, and reference to “a system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention. Conventional methods are used for the procedures described herein, such as those provided in the art, and demonstrated in the Examples and various general references. Unless otherwise stated, nucleic acid sequences described herein are given, when read from left to right, in the 5′ to 3′ direction. Nucleic acid sequences may be provided as DNA, as RNA, or a combination of DNA and RNA (e.g., a chimeric nucleic acid).

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both limits, ranges excluding either or both of those included limits are also included in the invention.

The term “and/or” used herein is to be taken as specific disclosure of each of the multiple specified features or components with or without another. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “about,” as applied to one or more values of interest, refers to a value that falls within 10%, 9%, 8%, 7%, 6%, 5%, 4%, 3%, 2%, 1%, or less in either direction (greater than or less than) of a stated reference value, unless otherwise stated or otherwise evident from the context (except where such number would exceed 100% of a possible value).

As used herein, the terms “binding affinity” or “dissociation constant” or “K_{d}” refer to the tendency of a molecule to bind (covalently or non-covalently) to a different molecule. A high K_{d} (which in the context of the present disclosure refers to blocked nucleic acid molecules binding to RNP2) indicates the presence of more unbound molecules, and a low K_{d} (which in the context of the present disclosure refers to unblocked nucleic acid molecules binding to RNP2) indicates the presence of more bound molecules. In the context of the present disclosure and the binding of blocked or unblocked nucleic acid molecules to RNP2, low K_{d} values are in a range from about 100 fM to about 1 aM or lower (e.g., 100 zM) and high K_{d} values are in the range of 100 nM-100 μM (10 mM) and thus are about 10⁵- to 10¹⁰-fold or higher as compared to low K_{d} values.

As used herein, the terms “binding domain” or “binding site” refer to a region on a protein, DNA, or RNA, to which specific molecules and/or ions (ligands) may form a covalent or non-covalent bond. By way of example, a polynucleotide sequence present on a nucleic acid molecule (e.g., a primer binding domain) may serve as a binding domain for a different nucleic acid molecule. Characteristics of binding sites are chemical specificity, a measure of the types of ligands that will bond, and affinity, which is a measure of the strength of the chemical bond.

As used herein, the terms “blocked nucleic acid molecule” or “blocked nucleic acid” refers to nucleic acid molecules that cannot bind to the first or second RNP complex to activate cis- or trans-cleavage. “Unblocked nucleic acid molecule” refers to a formerly blocked nucleic acid molecule that can bind to the second RNP complex (RNP2) to activate trans-cleavage of additional blocked nucleic acid molecules.

As used herein, the terms “cis-cleavage”, “cis-nucleic acid-guided nuclease activity”, “cis-cleavage activity”, “cis-mediated nucleic acid-guided nuclease activity”, “cis-nuclease activity”, “cis-mediated nuclease activity”, and variations thereof refer to sequence-specific cleavage of a target nucleic acid of interest, including an unblocked nucleic acid molecule, by a nucleic acid-guided nuclease in an RNP complex. Cis-cleavage is a single turn-over cleavage event in that only one substrate molecule is cleaved per event.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10, or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-ATCGAT-5′ is 100% complementary to a region of the nucleotide sequence 5′-GCTAGCTAG-3′.

As used herein, the term “contacting” refers to placement of two moieties in direct physical association, including in solid or liquid form. Contacting can occur in vitro with isolated cells (for example in a tissue culture dish or other vessel) or in samples or in vivo by administering an agent to a subject.

A “control” is a reference standard of a known value or range of values.

The terms “guide nucleic acid” or “guide RNA” or “gRNA” refer to a polynucleotide comprising 1) a crRNA region or guide sequence capable of hybridizing to the target strand of a target nucleic acid of interest, and 2) a scaffold sequence capable of interacting or complexing with a nucleic acid-guided nuclease. The crRNA region of the gRNA is a customizable component that enables specificity in every nucleic acid-guided nuclease reaction. A gRNA can include any polynucleotide sequence having sufficient complementarity with a target nucleic acid of interest to hybridize with the target nucleic acid of interest and to direct sequence-specific binding of a ribonucleoprotein (RNP) complex containing the gRNA and nucleic acid-guided nuclease to the target nucleic acid. A guide RNA may be from about 20 nucleotides to about 300 nucleotides long. Guide RNAs may be produced synthetically or generated from a DNA template.

“Modified” refers to a changed state or structure of a molecule. Molecules may be modified in many ways including chemically, structurally, and functionally. In one embodiment, a nucleic acid molecule (for example, a blocked nucleic acid molecule) may be modified by the introduction of non-natural nucleosides, nucleotides, and/or internucleoside linkages. In another embodiment, a modified protein (e.g., a modified or variant nucleic acid-guided nuclease) may refer to any polypeptide sequence alteration which is different from the wildtype.

The terms “nucleic acid-guided nuclease” or “CRISPR nuclease” refer to a CRISPR-associated protein that is an RNA-guided nucleic acid-guided nuclease suitable for assembly with a sequence-specific gRNA to form a ribonucleoprotein (RNP) complex.

The terms “percent sequence identity”, “percent identity”, or “sequence identity” refer to percent (%) sequence identity with respect to a reference polynucleotide or polypeptide sequence following alignment by standard techniques. Alignment for purposes of determining percent sequence identity can be achieved in various ways that are within the capabilities of one of skill in the art, for example, using publicly available computer software such as BLAST, BLAST-2, PSI-BLAST, or Megalign software. In some embodiments, the software is MUSCLE (Edgar, Nucleic Acids Res., 32(5):1792-1797 (2004)). Those skilled in the art can determine appropriate parameters for aligning sequences, including any algorithms needed to achieve maximal alignment over the full length of the sequences being compared. For example, in embodiments, percent sequence identity values are generated using the sequence comparison computer program BLAST (Altschul, et al., J. Mol. Biol., 215:403-410 (1990)).

As used herein, the terms “preassembled ribonucleoprotein complex”, “ribonucleoprotein complex”, “RNP complex”, or “RNP” refer to a complex containing a guide RNA (gRNA) and a nucleic acid-guided nuclease, where the gRNA is integrated with the nucleic acid-guided nuclease. The gRNA, which includes a sequence complementary to a target nucleic acid of interest, guides the RNP to the target nucleic acid of interest and hybridizes to it. The hybridized target nucleic acid-gRNA units are cleaved by the nucleic acid-guided nuclease. In the cascade assays described herein, a first ribonucleoprotein complex (RNP1) includes a first guide RNA (gRNA) specific to a target nucleic acid of interest, and a first nucleic acid-guided nuclease, such as, for example, cas12a or cas14a for a DNA target nucleic acid, or cas13a for an RNA target nucleic acid. A second ribonucleoprotein complex (RNP2) for signal boost includes a second guide RNA specific to an unblocked nucleic acid, and a second nucleic acid-guided nuclease, which may be different from or the same as the first nucleic acid-guided nuclease.

As used herein, the terms “protein” and “polypeptide” are used interchangeably. Proteins may or may not be made up entirely of amino acids.

As used herein, the term “sample” refers to tissues; cells or component parts; body fluids, including but not limited to peripheral blood, serum, plasma, ascites, urine, cerebrospinal fluid, sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. “Sample” may also refer to specimens or aliquots from food; agricultural products; pharmaceuticals; cosmetics, nutraceuticals; personal care products; environmental substances such as soil, water (from both natural and treatment sites), air, or sewer samples; industrial sites and products; and chemicals and compounds. A sample further may include a homogenate, lysate or extract. A sample further refers to a medium, such as a nutrient broth or gel, which may contain cellular components, such as proteins or nucleic acid molecules.

The terms “target DNA sequence”, “target sequence”, “target nucleic acid of interest”, “target molecule of interest”, “target nucleic acid”, or “target of interest” refer to any locus that is recognized by a gRNA sequence in vitro or in vivo. The “target strand” of a target nucleic acid of interest is the strand of the double-stranded target nucleic acid that is complementary to a gRNA. The spacer sequence of a gRNA may be 50%, 60%, 75%, 80%, 85%, 90%, 95%, 97.5%, 98%, 99% or more complementary to the target nucleic acid of interest. Optimal alignment can be determined with the use of any suitable algorithm for aligning sequences. Full complementarity is not necessarily required provided there is sufficient complementarity to cause hybridization and trans-cleavage activation of an RNP complex. A target nucleic acid of interest can include any polynucleotide, such as DNA (ssDNA or dsDNA) or RNA polynucleotides. A target nucleic acid of interest may be located in the nucleus or cytoplasm of a cell such as, for example, within an organelle of a eukaryotic cell, such as a mitochondrion or a chloroplast, or it can be exogenous to a host cell, such as a eukaryotic cell or a prokaryotic cell. The target nucleic acid of interest may be present in a sample, such as a biological or environmental sample, and it can be a viral nucleic acid molecule, a bacterial nucleic acid molecule, a fungal nucleic acid molecule, or a polynucleotide of another organism, such as a coding or a non-coding sequence, and it may include single-stranded or double-stranded DNA molecules, such as a cDNA or genomic DNA, or RNA molecules, such as mRNA, tRNA, and rRNA. The target nucleic acid of interest may be associated with a protospacer adjacent motif (PAM) sequence, which may include a 2-5 base pair sequence adjacent to the protospacer. In some embodiments 1, 2, 3, 4, 5, 6, 7, 8, 9, 10, or more target nucleic acids can be detected by the disclosed method.

As used herein, the terms “trans-cleavage”, “trans-cleavage activity”, “trans-nucleic acid-guided nuclease activity”, “trans-mediated nucleic acid-guided nuclease activity”, “trans-nuclease activity”, “trans-mediated nuclease activity” and variations thereof refer to indiscriminate, non-sequence-specific cleavage of a target nucleic acid molecule by a nucleic acid-guided nuclease (such as by a Cas12, Cas13, and Cas14) which is triggered by binding of N nucleotides of a target nucleic acid molecule to a gRNA and/or by cis- (sequence-specific) cleavage of a target nucleic acid molecule. Trans-cleavage is a “multiple turn-over” event, in that more than one substrate molecule is cleaved after initiation by a single turn-over cis-cleavage event.

Type V CRISPR/Cas nucleic acid-guided nucleases are a subtype of Class 2 CRISPR/Cas effector nucleases such as, but not limited to, engineered Cas12a, Cas12b, Cas12c, C2c4, C2c8, C2c5, C2c10, C2c9, CasX (Cas12e), CasY (Cas12d), Cas 13a nucleases or naturally-occurring proteins, such as a Cas12a isolated from, for example, Francisella tularensis subsp. novicida (Gene ID: 60806594), Candidatus methanoplasma termitum (Gene ID: 24818655), Candidatus methanomethylophilus alvus (Gene ID: 15139718), and [Eubacterium] eligens ATCC 27750 (Gene ID: 41356122), and an artificial polypeptide, such as a chimeric protein.

The term “variant” in the context of the present disclosure refers to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many if not most regions, identical. A variant and reference polypeptide may differ in one or more amino acid residues (e.g., substitutions, additions, and/or deletions). Variants include modifications—including chemical modifications—to one or more amino acids that do not involve amino acid substitutions, additions or deletions.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like.

### DETAILED DESCRIPTION

The present disclosure provides stabilization variant nucleases that have been engineered to substitute the amino acid serine for cysteine at various loci in the wildtype LbCas12a (Lachnospriaceae bacterium Cas12a) nuclease amino acid sequence so as to decrease or even prevent disulfide bridge formation and/or protein aggregation in solution. In the cascade assays described herein in which the stabilization variant nucleases are particularly useful, the stabilization variant nucleases promote enzyme and/or ribonucleoprotein stability for the cascade assay reagents.

Early and accurate detection and determination of infections and diseases is crucial for appropriate prevention strategies, accurate testing, confirmation, and further diagnosis and treatment. Nucleic acid-guided nucleases, such as the Cas12a and Cas13a endonucleases, can be utilized as diagnostic tools for the detection of target nucleic acids associated with diseases. However, currently available state-of-the-art CRISPR Cas12a-based nucleic acid detection relies on DNA amplification before using Cas12a enzymes, which significantly hinders the ability to perform rapid point-of-care testing. This is due to the fact that target-specific activation of Cas12a enzymes, referred herein as cis-cleavage, is a single turnover event in which the number of activated enzyme complexes is, at most, equal to the number of target nucleic acid copies in the sample. Once a ribonucleoprotein (RNP) complex is activated after completion of cis-cleavage, the RNP starts rapid non-specific trans-endonuclease activity. Some currently available methods use trans-cleavage to cleave fluorescent reporters that are initially quenched to generate a signal, thereby indicating the presence of a cis-cleavage event and thus the presence of the target nucleic acid. However, the K_{cat} of activated Cas12a complex is 17/sec and 3/sec for dsDNA and ssDNA targets, respectively. Therefore, for less than 10,000 target copies, the number of reporters cleaved is not sufficient to generate a signal in less than 60 minutes. Hence, all current technologies rely on DNA amplification to first generate billions of target copies to activate a proportional number of nucleic acid-guided nucleases to generate a detectable signal in 30-60 minutes. There is a need in the field to detect target nucleic acids (e.g., bacterial, viral, and fungal nucleic acid molecules) at a much faster rate for more efficient testing particularly for point-of-care and field testing.

The present disclosure describes stabilization variant nucleases that may be used in cascade assays to detect one or more target nucleic acids in a sample. The cascade assays provide a signal boost upon detection of the target nucleic acid(s) thereby affording rapid and accurate detection of one or more target nucleic acids in about 10 minutes or less. Signal boost utilizes two RNPs and reporter moieties able to reach attomolar (aM) detection (or lower) limits without the need to amplify the target nucleic acid, thus circumventing the complications of false positives produced from primer-dimerization, which usually occur in DNA amplification-based technologies when multiple primer sets are included in a single reaction. Moreover, since sequence-specific gRNAs are internalized into nucleic acid-guided nucleases to form preassembled RNPs, the disclosed methods further allow for accurate multiplex screening (e.g., syndromic testing) of a panel of target nucleic acids.

The stabilization variant nucleases disclosed herein are variants of wildtype Type V nuclease LbCas12a (Lachnospriaceae bacterium Cas12a), where one or more cysteine residues are substituted with serine residues, particularly cysteine residues which have been determined to be accessible to solvent when the variant nuclease is in solution. Wildtype LbCas12a has nine total residues, two of which—Cys965 and Cys1090—are highly accessible to solvent. Two other cysteine residues—Cys805 and Cys1116—are less accessible, and five cysteine residues—Cys10, Cys175, Cys565, Cys632 and Cys912—are largely inaccessible to solvent. These stabilization variant engineered nucleic acid-guided nucleases are particularly useful in embodiments of the cascade assay system described in U.S. Pat. Nos. 11,693,520; 11,702,686; 11,821,025; 11,820,983; and U.S. Ser. Nos. 17/861,207; 17/861,209; 18/208,272; 18,372,098; 18/078,821; 18/234,402; 18/078,031; 18/204,329 and 18/208,262, which utilize blocked nucleic acid molecules and in alternative embodiments, blocked primer molecules, which embodiment is also described in detail in these patents and published applications.

In an RNP with a single crRNA (i.e., that is, lacking a tracrRNA), Cas12a nucleases interact with a PAM (protospacer adjacent motif) sequence in a target nucleic acid for dsDNA unpairing and R-loop formation. Cas12a nucleases employ a multistep mechanism to ensure accurate recognition of spacer sequences in the target nucleic acid. The WED, REC1 and PAM-interacting (PI) domains of Cas12a nucleases are responsible for PAM recognition and for initiating invasion of the crRNA in the target dsDNA and for R-loop formation. It has been hypothesized that a conserved lysine residue is inserted into the dsDNA duplex, possibly initiating template strand/non-template strand unwinding. (See Jinek, et al, Mol. Cell, 73(3):589-600.e4 (2019).) PAM binding further introduces a kink in the target strand, which further contributes to local strand separation and facilitates base paring of the target strand to the seed segment of the crRNA while the displaced non-target strand is stabilized by interactions with the PAM-interacting domains. (Id.)

The cascade assays utilize a reaction mixture comprising: a first ribonucleoprotein (RNP1) complex containing a first nucleic acid-guided nuclease and a first guide RNA (gRNA) containing a sequence complementary to the target nucleic acid; a second ribonucleoprotein (RNP2) complex containing a second nucleic acid-guided nuclease, and a second gRNA that is not complementary to the target nucleic acid; a plurality of blocked nucleic acid molecules containing a sequence complementary to the second guide RNA (blocked nucleic acid molecules are not described in detail here, but see U.S. Pat. Nos. 11,693,520; 11,702,686; 11,821,025; 11,820,983; and U.S. Ser. Nos. 17/861,207; 17/861,209; 18/208,272; 18,372,098; 18/078,821; 18/234,402; 18/078,031; 18/204,329 and 18/208,262); and, optionally, reporter moieties comprising a signal moiety that is released by the trans-cleavage activity of activated RNP complexes wherein at least one of the first and/or second nucleic acid-guided nucleases is a stabilization variant nuclease. In this cascade assay, the blocked nucleic acid molecules cannot bind to the first or second RNP complex to activate trans-cleavage; however, once the blocked nucleic acid molecules are unblocked, they can bind to the second RNP complex (RNP2) to activate trans-cleavage.

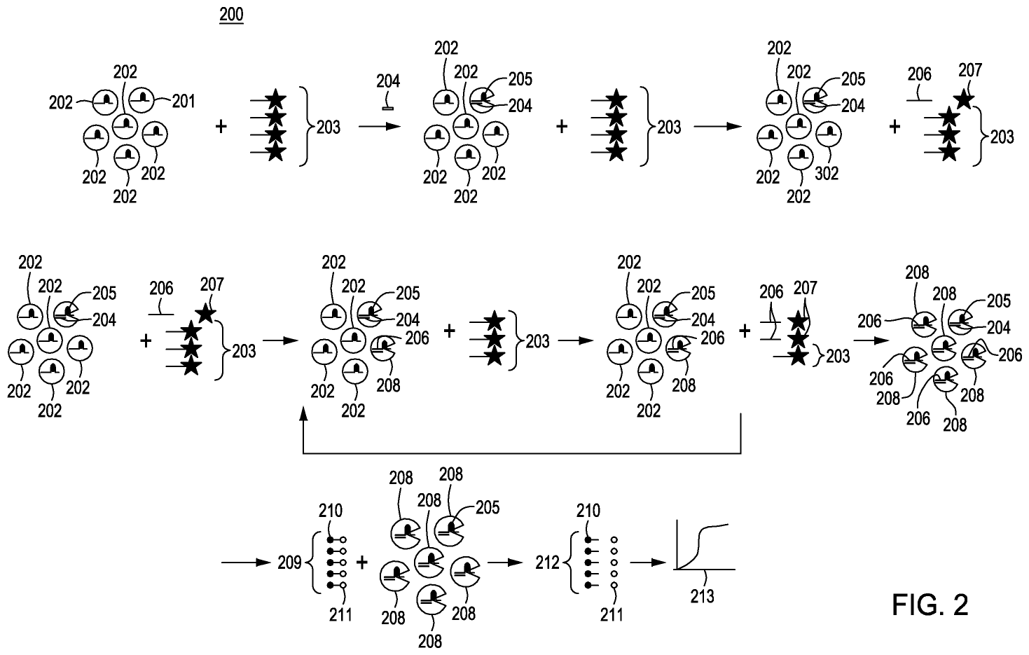

The cascade assay system is initiated when RNP1 binds to the target nucleic acid thereby activating RNP1 and generating both cis- and trans-cleavage activity. The cis-nuclease activity cuts the target nucleic acid, and trans-cleavage activity is initiated as well cleaving at least one of the blocked nucleic acid molecules to produce unblocked nucleic acid molecules. Upon binding of the unblocked nucleic acid molecule to the second gRNA in the RNP2 complex, RNP2 also is activated generating both cis- and trans-cleavage activity. As a result of the trans-cleavage activity of the RNP2 complex, at least one additional blocked nucleic acid molecule is converted to an unblocked nucleic acid molecule. Continued cleavage of blocked nucleic acid molecules and subsequent activation of more RNP2 complexes proceeds at an exponential rate. Additionally, a signal may be and preferably is generated upon trans-cleavage of a reporter moiety by the active RNP2 complexes; thus, a change in signal production indicates the presence of the target nucleic acid. In one embodiment such as shown in FIG. 2 (described in detail below), trans-cleavage by the first or second nucleic acid-guided nuclease (e.g., RNP1 or RNP2) may release a signal although the vast majority of the signal is the result of trans-cleavage by RNP2. In other embodiments, the reporter moiety may be bound to the blocked nucleic acid molecule, where trans-cleavage of the blocked nucleic acid molecules and conversion to unblocked nucleic acid molecules generates signal changes at rates that are directly proportional to the cleavage rate of the blocked nucleic acid molecules, thus allowing for real time reporting of results.

In short, the blocked nucleic acid molecules serve as a gatekeeper for preventing errant activation of RNP2. Only upon binding of the target nucleic acid to RNP1 are the blocked nucleic acid molecules unblocked, making them available to activate RNP2.

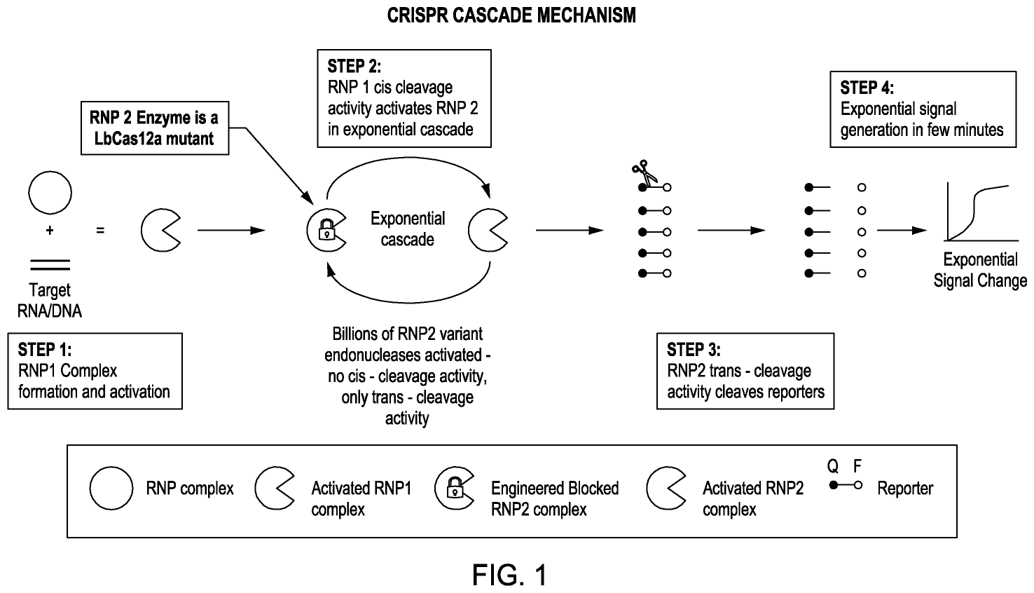

FIG. 1 is a simplified diagram of a cascade assay reaction in which the stabilization variant nuclease described herein may be employed. FIG. 1 at left (step 1) illustrates a first preassembled ribonucleoprotein complex (RNP1) where RNP1 comprises first nucleic acid-guided nucleases and first guide RNAs (gRNAs) specific to target nucleic acid(s) of interest. The first nucleic acid-guided nuclease may be, for example, Cas12a or Cas14a for a DNA target, or Cas13a for an RNA target. Also seen at left is a target nucleic acid (RNA or DNA). When the target nucleic acid binds to the first gRNA in RNP1, RNP1 is activated and cis-cleavage of the target nucleic acid occurs, initiating trans-cleavage of other nucleic acids in the sample. Moving right (step 2), a “locked” RNP2 complex is seen, where the locked RNP2 complex comprises a second nucleic acid-guided nuclease and a second guide RNA (gRNA). Either one or both of the first and second nucleic acid-guided nucleases may be a stability variant nuclease as described herein.

In addition to RNP1, RNP2 and the target nucleic acid, also present in the reaction mixture are blocked nucleic acid molecules. Blocked nucleic acid molecules are nucleic acid molecules that cannot bind to either the RNP1 or RNP2 complexes to activate cis- or trans-cleavage. The blocked nucleic acid molecules do not bind to RNP1 due to sequence incompatibility with the first gRNA. And although the blocked nucleic acid molecules do possess sequence compatibility with the gRNAs in RNP2 (i.e., the gRNA2s), the blocked nucleic acid molecules have been configured so that they cannot act as a substrate for RNP2 processing until they are unblocked. Only upon binding of the target nucleic acid to RNP1 and the triggering of trans-cleavage activity are the blocked nucleic acid molecules unblocked—thereby providing single stranded nucleic acids that are sequence compatible with the gRNA in RNP2. The unblocked single stranded nucleic acids then activate RNP2. The activated RNP2 complexes trigger further trans-cleavage, and more blocked nucleic acid molecules are converted to unblocked nucleic acid molecules which then activate more RNP2 complexes, providing exponential cleavage of blocked nucleic acid molecules and RNP2 formation and activation.

Also present in the reactions are reporter moieties. Here, the reporter moieties are illustrated as separate from the RNP2 complex. The reporter moieties may be synthetic molecules linked or conjugated to a reporter and quencher such as, for example, a TaqMan probe with a dye label (FAM) on the 5′ end and a minor groove binder (MGB) and a quencher on the 3′ end. The reporter and quencher can be about 20-30 bases apart or less for effective quenching via fluorescence resonance energy transfer (FRET). Signal generation, however, may occur through different mechanisms. Other detectable moieties, labels or reporters can also be used to detect a target nucleic acid. Reporter moieties can be labeled in a variety of ways, including the direct or indirect attachment of a detectable moiety such as a fluorescent moiety, hapten, colorimetric moiety and the like.

Examples of detectable moieties include various radioactive moieties, enzymes, prosthetic groups, fluorescent markers, luminescent markers, bioluminescent markers, metal particles, protein-protein binding pairs, protein-antibody binding pairs and the like. Examples of fluorescent moieties include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, cyanines, dansyl chloride, phycocyanin, phycoerythrin and the like. Examples of bioluminescent markers include, but are not limited to, luciferase (e.g., bacterial, firefly, click beetle and the like), luciferin, acquorin and the like. Examples of enzyme systems having visually detectable signals include, but are not limited to, galactosidases, glucuronidases, phosphatases, peroxidases, cholinesterases and the like. Identifiable markers also include radioactive compounds such as ¹²⁵I, ³⁵S, ¹⁴C, or ³H.

The trans-cleavage triggered by the activation of RNP2 complexes in the cascade cleaves the, e.g., fluorescent reporters that are initially quenched to generate a signal in step 3. The configuration of the reporter moieties may be as shown in FIG. 1, or the blocked nucleic acid molecules may be linked to single-strand or double-strand DNA or RNA reporter moieties, such that the signal change is detected proportional to the cleavage rate, which increases with every new activated RNP2 complex over time. In step 4, exponential signal generation is achieved in only a few minutes.

Again, for detailed information regarding several embodiments of blocked nucleic acid molecules of use in the cascade assay system, see U.S. Pat. Nos. 11,693,520; 11,702,686; 11,821,025; 11,820,983; and U.S. Ser. Nos. 17/861,207; 17/861,209; 18/208,272; 18,372,098; 18/078,821; 18/234,402; 18/078,031; 18/204,329 and 18/208,262.

FIG. 1, described above, depicts the cascade assay generally. A specific embodiment of the cascade assay utilizing blocked nucleic acid molecules is depicted in FIG. 2 and described in detail below. In this embodiment, a blocked nucleic acid molecule is used to prevent the activation of RNP2 in the absence of a target nucleic acid of interest. The method in FIG. 2 begins with providing the cascade assay components RNP1 (201), RNP2 (202) and blocked nucleic acid molecules (203). RNP1 (201) comprises a gRNA specific for a target nucleic acid of interest and a nucleic acid-guided nuclease (e.g., Cas 12a or Cas 14 for a DNA target nucleic acid of interest or a Cas 13a for an RNA target nucleic acid of interest) and RNP2 (202) comprises a gRNA specific for an unblocked nucleic acid molecule and a second nucleic acid-guided nuclease. The nucleic acid-guided nucleases in RNP1 (201) and RNP2 (202) may be the same or different, depending on whether the target nucleic acid of interest and the unblocked nucleic acid molecule comprise DNA or RNA, and may both be stabilization variant nucleases. It is key, however, that the first and second nucleic acid-guided nucleases in RNP1 and RNP2, respectively, may be activated to have trans-cleavage activity following binding of a target nucleic acid of interest or an unblocked nucleic acid molecule and/or initiation of cis-cleavage activity.

In a first step, a sample comprising a target nucleic acid of interest (204) is added to the cascade assay reaction mix. The target nucleic acid of interest (204) combines with and activates RNP1 (205) but does not interact with or activate RNP2 (202). Once activated, RNP1 binds the target nucleic acid of interest (204) and cuts the target nucleic acid of interest (204) via sequence-specific cis-cleavage, there the binding of the target nucleic acid of interest and/or the initiation of cis-cleavage activity activates non-specific trans-cleavage of other nucleic acids present in the reaction mix, including the blocked nucleic acid molecules (203). At least one of the blocked nucleic acid molecules (203) becomes an unblocked nucleic acid molecule (206) when the blocking moiety (207) is removed. As described below, “blocking moiety” may refer to nucleoside modifications, topographical configurations such as secondary structures, and/or structural modifications.

Once at least one of the blocked nucleic acid molecules (203) is unblocked, the unblocked nucleic acid molecule (206) can then bind to and activate an RNP2 (208). Because the nucleic acid-guided nucleases in the RNP1s (205) and RNP2s (208) have both cis- and trans-cleavage activity, the trans-cleavage activity causes more blocked nucleic acid molecules (203) become unblocked nucleic acid molecules (206) triggering activation of even more RNP2s (208) and more trans-cleavage activity in a cascade. FIG. 2 at bottom depicts the concurrent activation of reporter moieties. Intact reporter moieties (209) comprise a quencher (210) and a fluorophore (211) linked by a nucleic acid sequence. As described above in relation to FIG. 1, the reporter moieties are also subject to trans-cleavage by activated RNP1 (205) and RNP2 (208). The intact reporter moieties (209) become activated reporter moieties (212) when the quencher (210) is separated from the fluorophore (211), emitting a fluorescent signal (213). Signal strength increases rapidly as more blocked nucleic acid molecules (203) become unblocked nucleic acid molecules (206) triggering cis-cleavage activity of more RNP2s (208) and thus more trans-cleavage activity of the reporter moieties (209). Again, the reporter moieties are shown here as separate molecules from the blocked nucleic acid molecules, but other configurations may be employed. One particularly advantageous feature of the cascade assay is that, with the exception of the gRNA in the RNP1 (gRNA1), the cascade assay components are modular in the sense that the components may stay the same no matter what target nucleic acid(s) of interest are being detected.

A blocked nucleic acid molecule may be single-stranded or double-stranded, circular or linear, and may further contain a partially hybridized nucleic acid sequence containing cleavable secondary loop structures. Such blocked nucleic acid molecules typically have a low binding affinity, or high dissociation constant (K_{d}) in relation to binding to RNP2 and may be referred to herein as a high K_{d} nucleic acid molecule. In the context of the present disclosure, the binding of blocked or unblocked nucleic acid molecules to RNP2, low K_{d} values range from about 100 fM to about 1 aM or lower (e.g., 100 zM) and high K_{d} values are in the range of 100 nM to about 10-100 10 mM and thus are about 10⁵-, 10⁶-, 10⁷-, 10⁸-, 10⁹- to 10¹⁰-fold or higher as compared to low K_{d} values. Of course, the ideal blocked nucleic acid molecule would have an “infinite K_{d}.”

The blocked nucleic acid molecules (high K_{d} molecules) described herein can be converted into unblocked nucleic acid molecules (low K_{d} molecules—also in relation to binding to RNP2) via cleavage of nuclease-cleavable regions (i.e., via active RNP1s and RNP2s). The unblocked nucleic acid molecule has a higher binding affinity for the gRNA in RNP2 than does the blocked nucleic acid molecule.

Once the unblocked nucleic acid molecule is bound to RNP2, RNP2 activation triggers trans-cleavage activity, which in turn leads to more RNP2 activation by further cleaving blocked nucleic acid molecules, resulting in a positive feedback loop or cascade.

In embodiments where blocked nucleic acid molecules are linear and/or form a secondary structure, the blocked nucleic acid molecules may be single-stranded (ss) or double-stranded (ds) and contain a first nucleotide sequence and a second nucleotide sequence. The first nucleotide sequence has sufficient complementarity to hybridize to a gRNA of RNP2 (i.e., gRNA2), and the second nucleotide sequence does not. The first and second nucleotide sequences of a blocked nucleic acid molecule may be on the same nucleic acid molecule (e.g., for single-strand embodiments) or on separate nucleic acid molecules (e.g., for double-strand embodiments). Trans-cleavage (e.g., via RNP1 or RNP2) of the second nucleotide sequence converts the blocked nucleic acid molecule to a single-strand unblocked nucleic acid molecule. The unblocked nucleic acid molecule contains only the first nucleotide sequence, which has sufficient complementarity to hybridize to the gRNA of RNP2, thereby activating the trans-cleavage activity of RNP2.

In some embodiments, the second nucleotide sequence at least partially hybridizes to the first nucleotide sequence, resulting in a secondary structure containing at least one loop (e.g., hairpin loops, tetraloops, pseudoknots, junctions, kissing hairpins, internal loops, bulges, and multibranch loops). Such loops block the nucleic acid molecule from binding or incorporating into an RNP complex thereby initiating cis- or trans-cleavage. Again, see U.S. Pat. Nos. 11,693,520; 11,702,686; 11,821,025; 11,820,983; and U.S. Ser. Nos. 17/861,207; 17/861,209; 18/208,272; 18,372,098; 18/078,821; 18/234,402; 18/078,031; 18/204,329 and 18/208,262.

In some embodiments, the blocked nucleic acid molecule may contain a protospacer adjacent motif (PAM) sequence, or partial PAM sequence, positioned between the first and second nucleotide sequences, where the first sequence is 5′ to the PAM sequence, or partial PAM sequence. Inclusion of a PAM sequence may increase the reaction kinetics internalizing the unblocked nucleic acid molecule into RNP2 and thus decrease the time to detection. In other embodiments, the blocked nucleic acid molecule does not contain a PAM sequence.

Nucleotide mismatches can be introduced into the regions of the blocked nucleic acid regions containing double-strand segments to reduce the melting temperature (Tₘ) of the segment such that once the loop (L) is cleaved, the double-strand segment is unstable and dehybridizes rapidly. The percentage of nucleotide mismatches of a given segment may vary between 0% and 50%; however, the maximum number of nucleotide mismatches is limited to a number where the secondary loop structure still forms. “Segments” in the above statement refers to generally double-strand region of the blocked nucleic acid molecules. In other words, the number of hybridized bases can be less than or equal to the length of each double-strand segment and vary based on number of mismatches introduced.

In any of the foregoing embodiments, the blocked nucleic acid molecules of the disclosure may further contain a reporter moiety attached thereto such that cleavage of the blocked nucleic acid releases a signal from the reporter moiety.

Also, in any of the foregoing embodiments, the blocked nucleic acid molecule may be a modified or non-naturally occurring nucleic acid molecule. In some embodiments, the blocked nucleic acid molecules of the disclosure may further contain a locked nucleic acid (LNA), a bridged nucleic acid (BNA), and/or a peptide nucleic acid (PNA). The blocked nucleic acid molecule may contain a modified or non-naturally occurring nucleoside, nucleotide, and/or internucleoside linkage, such as a 2′-O-methyl (2′-O-Me) modified nucleoside, a 2′-fluoro (2′-F) modified nucleoside, and a phosphorothioate (PS) bond, any other nucleic acid molecule modifications described above, and any combination thereof.

In some embodiments, the blocked nucleic acid molecules provided herein are circular DNAs, RNAs or chimeric (DNA-RNA) molecules, and the blocked nucleic acid molecules may include different base compositions. For the circular design of blocked nucleic acid molecules, the 5′ and 3′ ends are covalently linked together. In some embodiments, the 5′ and 3′ portions are hybridized to each other using DNA, RNA, LNA, BNA, or PNA bases which have a very high melting temperature (Tₘ). The high Tₘ causes the structure to effectively behave as a circular molecule even though the 5′ and 3′ ends are not covalently linked. The 5′ and 3′ ends can also have base non-naturally occurring modifications such as phosphorothioate bonds to provide increased stability. This configuration makes internalization of the blocked nucleic acid molecule into RNP2—and subsequent RNP2 activation—sterically unfavorable, thereby blocking the progression of the cascade assay. Thus, RNP2 activation (e.g., trans-cleavage activity) happens after cleavage of a portion of the blocked nucleic acid molecule followed by linearization and internalization of unblocked nucleic acid molecule into RNP2.

### Stabilized Variant Nucleases

The stabilization variant nucleic acid-guided nucleases described herein are used in CRISPR-based cascade assays to detect one or more target nucleic acids in a sample. The cascade assays provide signal boost upon detection of target nucleic acids without amplification of the target nucleic acids. The stabilization variant nucleases are particularly useful in point-of-care applications, in the field, and as components of assay kits.

Table 1 shows the amino acid sequences for wildtype LbCas12a (SEQ ID NO: 1) and the stabilization variant LbCas12a nucleases described herein. For SEQ ID NO: 1 (LbCas12a wildtype) nine cysteine amino acid residues are bolded and underlined. For SEQ ID NOs: 2-12 (the stabilization variant LbCas12a nucleases) the cysteine residue(s) that have been substituted with serine are bolded and underlined. Table 2 provides a value for the accessibility to solvent (scale 0 to 100) for the nine cysteine residues. Amino acids with low values (i.e., 0.6) are buried inside of the protein, which amino acids with high values (i.e., 71.7) are exposed to the surface.

TABLE 1

| Species | | |
|---|---|---|
| Name | SEQ | |
| and | ID | |
| Variant | NO: | Protein Sequence |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 1 | NKELENLEINLRKEIAKAFKGNEGYKSLFKKDIIETILPEFLDDKDE |
| wildtype | | IALVNSFNGFTTAFTGFFDNRENMFSEEAKSTSIAFRCINENLTRYI |
| (LbCas12a) | | SNMDIFEKVDAIFDKHEVQEIKEKILNSDYDVEDFFEGEFFNFVLT |
| PDD: | | QEGIDVYNAIIGGFVTESGEKIKGLNEYINLYNQKTKQKLPKFKPL |
| 6KL9_A | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEV |
| | | RVLLKHDDNPYVIGIDRGERNLLYIVVVDGKGNIVEQYSLNEIINN |
| | | FNGIRIKTDYHSLLDKKEKERFEARQNWTSIENIKELKAGYISQVV |
| | | HKICELVEKYDAVIALEDLNSGFKNSRVKVEKQVYQKFEKMLID |
| | | KLNYMVDKKSNPCATGGALKGYQITNKFESFKSMSTQNGFIFYIP |
| | | AWLTSKIDPSTGFVNLLKTKYTSIADSKKFISSFDRIMYVPEEDLFE |
| | | FALDYKNFSRTDADYIKKWKLYSYGNRIRIFRNPKKNNVFDWEE |
| | | VCLTSAYKELFNKYGINYQQGDIRALLCEQSDKAFYSSFMALMSL |
| | | MLQMRNSITGRTDVDFLISPVKNSDGIFYDSRNYEAQENAILPKN |
| | | ADANGAYNIARKVLWAIGQFKKAEDEKLDKVKIAISNKEWLEYA |
| | | QTSVKH |
| Lachnospiraceae | SEQ | MSKLEKFTNSYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 2 | NKELENLEINLRKEIAKAFKGNEGYKSLFKKDIIETILPEFLDDKDE |
| C10S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPCATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 3 | NKELENLEINLRKEIAKAFKGNEGYKSLFKKDIIETILPEFLDDKDE |
| C175S | | IALVNSFNGFTTAFTGFFDNRENMFSEEAKSTSIAFRSINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPCATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 4 | NKELENLEINLRKEIAKAFKGNEGYKSLFKKDIIETILPEFLDDKDE |
| C565S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKSLQKIDKDDVNGNYEKINYKLLPGPNKML |
| | | PKVFFSKKWMAYYNPSEDIQKIYKNGTFKKGDMFNLNDCHKLID |
| | | FFKDSISRYPKWSNAYDFNFSETEKYKDIAGFYREVEEQGYKVSF |
| | | ESASKKEVDKLVEEGKLYMFQIYNKDFSDKSHGTPNLHTMYFKL |
| | | LFDENNHGQIRLSGGAELFMRRASLKKEELVVHPANSPIANKNPD |
| | | NPKKTTTLSYDVYKDKRFSEDQYELHIPIAINKCPKNIFKINTEVRV |
| | | LLKHDDNPYVIGIDRGERNLLYIVVVDGKGNIVEQYSLNEIINNFN |
| | | GIRIKTDYHSLLDKKEKERFEARQNWTSIENIKELKAGYISQVVHK |
| | | ICELVEKYDAVIALEDLNSGFKNSRVKVEKQVYQKFEKMLIDKLN |
| | | YMVDKKSNPCATGGALKGYQITNKFESFKSMSTQNGFIFYIPAWL |
| | | TSKIDPSTGFVNLLKTKYTSIADSKKFISSFDRIMYVPEEDLFEFAL |
| | | DYKNFSRTDADYIKKWKLYSYGNRIRIFRNPKKNNVFDWEEVCL |
| | | TSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 5 | NKELENLEINLRKEIAKAFKGNEGYKSLFKKDIIETILPEFLDDKDE |
| C632S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDSHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPCATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 6 | NKELENLEINLRKEIAKAFKGNEGYKSLFKKDIIETILPEFLDDKDE |
| C805S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKSPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPCATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 7 | NKELENLEINLRKEIAKAFKGNEGYKSLFKKDIIETILPEFLDDKDE |
| C912S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KISELVEKYDAVIALEDLNSGFKNSRVKVEKQVYQKFEKMLIDKL |
| | | NYMVDKKSNPCATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 8 | NKELENLEINLRKEIAKAFKGNEGYKSLFKKDIIETILPEFLDDKDE |
| C965S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPSATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: 9 | NKELENLEINLRKEIAKAFKGNEGYKSLFKKDIIETILPEFLDDKDE |
| C1090S | | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPSATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVS |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: | NKELENLEINLRKEIAKAFKGNEGYKSLFKKDIIETILPEFLDDKDE |
| C1116S | 10 | IALVNSFNGFTTAFTGFFDNRENMFSEEAKSTSIAFRCINENLTRYI |
| | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPCATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVC |
| | | LTSAYKELFNKYGINYQQGDIRALLSEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: | NKELENLEINLRKEIAKAFKGNEGYKSLFKKDIIETILPEFLDDKDE |
| C965S/ | 11 | IALVNSFNGFTTAFTGFFDNRENMFSEEAKSTSIAFRCINENLTRYI |
| C1090S | | SNMDIFEKVDAIFDKHEVQEIKEKILNSDYDVEDFFEGEFFNFVLT |
| | | QEGIDVYNAIIGGFVTESGEKIKGLNEYINLYNQKTKQKLPKFKPL |
| | | YKQVLSDRESLSFYGEGYTSDEEVLEVFRNTLNKNSEIFSSIKKLE |
| | | KLFKNFDEYSSAGIFVKNGPAISTISKDIFGEWNVIRDKWNAEYDD |
| | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKCLQKIDKDDVNGNYEKINYKLLPGPNKM |
| | | LPKVFFSKKWMAYYNPSEDIQKIYKNGTFKKGDMFNLNDCHKLI |
| | | DFFKDSISRYPKWSNAYDFNFSETEKYKDIAGFYREVEEQGYKVS |
| | | FESASKKEVDKLVEEGKLYMFQIYNKDFSDKSHGTPNLHTMYFK |
| | | LLFDENNHGQIRLSGGAELFMRRASLKKEELVVHPANSPIANKNP |
| | | DNPKKTTTLSYDVYKDKRFSEDQYELHIPIAINKCPKNIFKINTEVR |
| | | VLLKHDDNPYVIGIDRGERNLLYIVVVDGKGNIVEQYSLNEIINNF |
| | | NGIRIKTDYHSLLDKKEKERFEARQNWTSIENIKELKAGYISQVVH |
| | | KICELVEKYDAVIALEDLNSGFKNSRVKVEKQVYQKFEKMLIDKL |
| | | NYMVDKKSNPSATGGALKGYQITNKFESFKSMSTQNGFIFYIPAW |
| | | LTSKIDPSTGFVNLLKTKYTSIADSKKFISSFDRIMYVPEEDLFEFA |
| | | LDYKNFSRTDADYIKKWKLYSYGNRIRIFRNPKKNNVFDWEEVS |
| | | LTSAYKELFNKYGINYQQGDIRALLCEQSDKAFYSSFMALMSLML |
| | | QMRNSITGRTDVDFLISPVKNSDGIFYDSRNYEAQENAILPKNADA |
| | | NGAYNIARKVLWAIGQFKKAEDEKLDKVKIAISNKEWLEYAQTS |
| | | VKH |
| Lachnospiraceae | SEQ | MSKLEKFTNCYSLSKTLRFKAIPVGKTQENIDNKRLLVEDEKRAE |
| bacterium | ID | DYKGVKKLLDRYYLSFINDVLHSIKLKNLNNYISLFRKKTRTEKE |
| Cas12a | NO: | NKELENLEINLRKEIAKAFKGNEGYKSLFKKDIIETILPEFLDDKDE |
| C565S/ | 12 | IALVNSFNGFTTAFTGFFDNRENMFSEEAKSTSIAFRCINENLTRYI |
| C805S/ | | SNMDIFEKVDAIFDKHEVQEIKEKILNSDYDVEDFFEGEFFNFVLT |
| C912S/ | | QEGIDVYNAIIGGFVTESGEKIKGLNEYINLYNQKTKQKLPKFKPL |
| C965S/ | | YKQVLSDRESLSFYGEGYTSDEEVLEVFRNTLNKNSEIFSSIKKLE |
| C1090S/ | | KLFKNFDEYSSAGIFVKNGPAISTISKDIFGEWNVIRDKWNAEYDD |
| C1116S | | IHLKKKAVVTEKYEDDRRKSFKKIGSFSLEQLQEYADADLSVVEK |
| | | LKEIIIQKVDEIYKVYGSSEKLFDADFVLEKSLKKNDAVVAIMKDL |
| | | LDSVKSFENYIKAFFGEGKETNRDESFYGDFVLAYDILLKVDHIYD |
| | | AIRNYVTQKPYSKDKFKLYFQNPQFMGGWDKDKETDYRATILRY |
| | | GSKYYLAIMDKKYAKSLQKIDKDDVNGNYEKINYKLLPGPNKML |
| | | PKVFFSKKWMAYYNPSEDIQKIYKNGTFKKGDMFNLNDCHKLID |
| | | FFKDSISRYPKWSNAYDFNFSETEKYKDIAGFYREVEEQGYKVSF |
| | | ESASKKEVDKLVEEGKLYMFQIYNKDFSDKSHGTPNLHTMYFKL |
| | | LFDENNHGQIRLSGGAELFMRRASLKKEELVVHPANSPIANKNPD |
| | | NPKKTTTLSYDVYKDKRFSEDQYELHIPIAINKSPKNIFKINTEVRV |
| | | LLKHDDNPYVIGIDRGERNLLYIVVVDGKGNIVEQYSLNEIINNFN |
| | | GIRIKTDYHSLLDKKEKERFEARQNWTSIENIKELKAGYISQVVHK |
| | | ISELVEKYDAVIALEDLNSGFKNSRVKVEKQVYQKFEKMLIDKLN |
| | | YMVDKKSNPSATGGALKGYQITNKFESFKSMSTQNGFIFYIPAWL |
| | | TSKIDPSTGFVNLLKTKYTSIADSKKFISSFDRIMYVPEEDLFEFAL |
| | | DYKNFSRTDADYIKKWKLYSYGNRIRIFRNPKKNNVFDWEEVSL |
| | | TSAYKELFNKYGINYQQGDIRALLSEQSDKAFYSSFMALMSLMLQ |
| | | MRNSITGRTDVDFLISPVKNSDGIFYDSRNYEAQENAILPKNADAN |
| | | GAYNIARKVLWAIGQFKKAEDEKLDKVKIAISNKEWLEYAQTSV |
| | | KH |

TABLE 2

| Variant | SEQ ID NO: | Solvent Accessibility Value |
|---|---|---|
| C10S | 2 | 10.9 |
| C175S | 3 | 0.6 |
| C565S | 4 | 8.9 |
| C632S | 5 | 0 |
| C805S | 6 | 20.4 |
| C912S | 7 | 5.8 |
| C965S | 8 | 64.6 |
| C1090S | 9 | 71.7 |
| C1116S | 10 | 20.7 |

### Uses of the CRISPR Assays and Arrays

The present disclosure describes cascade assays for detecting several to many to a massively multiplexed number of target nucleic acids of interest in a sample that provide instantaneous or nearly instantaneous results in less than ten minutes, have a minimum workflow, and provide accurate results at low cost. The various embodiments of the cascade assay are notable in that, with the exception of the gRNAs in RNP1, the cascade assay components may stay the same no matter what target nucleic acid(s) of interest are being detected. As described above, the cascade assay can be massively multiplexed for detecting several to many to a massively multiplexed number of target nucleic acid molecules simultaneously without amplification of the nucleic acids in the sample.

As described above, early and accurate identification of, e.g., infectious agents, microbe contamination, and variant nucleic acid sequences that indicate the presence of such diseases such as cancer or contamination by heterologous sources is important in order to select correct therapeutic treatment, identify tainted food, pharmaceuticals, cosmetics and other commercial goods; and to monitor the environment. The cascade assay described herein can be applied in diagnostics for, e.g., infectious disease (including but not limited to Covid, HIV, flu, the common cold, Lyme disease, STDs, chicken pox, diptheria, mononucleosis, hepatitis, UTIs, pneumonia, tetanus, rabies, malaria, dengue fever, Ebola, plague), for rapid liquid biopsies and companion diagnostics (biomarkers for cancers, early detection, progression, monitoring), prenatal testing (including but not limited to chromosomal abnormalities and genetic diseases such as sickle cell, including over-the-counter versions of prenatal testing assays), rare disease testing (achondroplasia, Addison's disease, α1-antitrypsin deficiency, multiple sclerosis, muscular dystrophy, cystic fibrosis, blood factor deficiencies), SNP detection/DNA profiling/epigenetics, genotyping, low abundance transcript detection, library quantification of NGS, screening biologics (including engineered therapeutic cells for genetic integrity and/or contamination), development of agricultural products, food compliance testing and quality control (e.g., detection of genetically modified products, confirmation of source for high value commodities, contamination detection), infectious disease in livestock, infectious disease in cash crops, livestock breeding, drug screening, personal genome testing including clinical trial stratification, personalized medicine, nutrigenomics, drug development and drug therapy efficacy, transplant compatibility and monitoring, environmental testing and forensics, and bioterrorism agent monitoring.

The target nucleic acids of interest are derived from samples as described in more detail above. Suitable samples for testing include, but are not limited to, any environmental sample, such as air, water, soil, surface, food, clinical sites and products, industrial sites and products, pharmaceuticals, medical devices, nutraceuticals, cosmetics, personal care products, agricultural equipment and sites, and commercial samples, and any biological sample obtained from an organism or a part thereof, such as a plant, animal, or microbe. In some embodiments, the biological sample is obtained from an animal subject, such as a human subject. A biological sample may be any solid or fluid sample obtained from, excreted by or secreted by any living organism, including, without limitation, single celled organisms, such as bacteria, yeast, protozoans, and amoebas among others, multicellular organisms including plants or animals, including samples from a healthy or apparently healthy human subject or a human patient affected by a condition or disease to be diagnosed or investigated, such as an infection with a pathogenic microorganism, such as a pathogenic bacteria or virus.

For example, a biological sample can be a biological fluid obtained from a human or non-human (e.g., livestock, pets, wildlife) animal, and may include but is not limited to blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface (e.g., a nasal or buccal swab).

The sample can be a viral or bacterial sample or a biological sample that has been minimally processed as described herein, e.g., only treated with a brief lysis step prior to detection. In other embodiments, minimal processing can include thermal lysis at an elevated temperature. In some embodiments, minimal processing can include treating the sample with chaotropic salts such as guanidine isothiocyanate or guanidine HCl and in some embodiments, minimal processing may include contacting the sample with reducing agents such as DTT or TCEP and EDTA to inactivate inhibitors and/or other nucleases present in the samples. In other embodiments, minimal processing for biofluids may include centrifuging the samples to obtain cell-debris free supernatant before applying the reagents.

The components of the cascade assay may be provided in various kits for testing at, e.g., point-of-care facilities, in the field, pandemic testing sites, and the like. In one aspect, the kit for detecting target nucleic acids of interest in a sample includes: several to many to massively multiplexed first ribonucleoprotein complexes (RNP1s, targeting one to several to many to a massively multiplexed number of different target nucleic acid molecules of interest), second ribonucleoprotein complexes (RNP2s), blocked nucleic acid molecules, and reporter moieties, where one or both of the first and/or second nucleic acid-guided nucleases is a stabilization variant nuclease. The dual RNP1-1/RNP1-2 comprises an RNP1-1 domain and an RNP1-2 domain. The RNP1-1 domains comprises gRNA1-1s, where the gRNA1-1s include the sequences complementary to the target nucleic acids of interest.

The kits described herein may further include a sample collection device, e.g., a syringe, lancet, nasal swab, or buccal swab for collecting a biological sample from a subject, and/or a sample preparation reagent, e.g., a lysis reagent. The kits can be used with an instrument comprising, e.g., a sample prep module and a detection module. Each component of the kit may be in a separate container or two or more components may be in the same container. In addition, the kit may further include instructions for use and other information.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

I claim:

1. A stabilization variant LbCas12a nuclease having a sequence comprising any one of SEQ ID NOs: 2-12.

2. The stabilization variant LbCas12a nuclease of claim 1 comprising the sequence SEQ ID NO: 2.

3. The stabilization variant LbCas12a nuclease of claim 1 comprising the sequence SEQ ID NO: 3.

4. The stabilization variant LbCas12a nuclease of claim 1 comprising the sequence SEQ ID NO: 4.

5. The stabilization variant LbCas12a nuclease of claim 1 comprising the sequence SEQ ID NO: 5.

6. The stabilization variant LbCas12a nuclease of claim 1 comprising the sequence SEQ ID NO: 6.

7. The stabilization variant LbCas12a nuclease of claim 1 comprising the sequence SEQ ID NO: 7.

8. The stabilization variant LbCas12a nuclease of claim 1 comprising the sequence SEQ ID NO: 8.

9. The stabilization variant LbCas12a nuclease of claim 1 comprising the sequence SEQ ID NO: 9.

10. The stabilization variant LbCas12a nuclease of claim 1 comprising the sequence SEQ ID NO: 10.

11. The stabilization variant LbCas12a nuclease of claim 1 comprising the sequence SEQ ID NO: 11.

12. The stabilization variant LbCas12a nuclease of claim 1 comprising the sequence SEQ ID NO: 12.

13. A reaction mixture comprising:
a first ribonucleoprotein (RNP) complex (RNP1) comprising a first nucleic acid-guided nuclease and a first guide RNA (gRNA); wherein the first gRNA comprises a sequence complementary to a target nucleic acid of interest, and wherein the first nucleic acid-guided nuclease exhibits both cis-cleavage activity and trans-cleavage activity;
a second ribonucleoprotein complex (RNP2) comprising a second nucleic acid-guided nuclease and a second gRNA that is not complementary to the target nucleic acid of interest; wherein the second nucleic acid-guided nuclease exhibits both cis-cleavage activity and trans-cleavage activity; and
a plurality of blocked nucleic acid molecules comprising a sequence complementary to the second gRNA, wherein the blocked nucleic acid molecules cannot activate the RNP1 or the RNP2, and wherein at least one of the first and second nucleic acid-guided nucleases is the stabilization variant LbCas12a nuclease of claim 1.

14. The reaction mixture of claim 13, wherein the plurality of blocked nucleic acid molecules comprises:
a first region, which is the sequence complementary to the second gRNA;
one or more second regions not complementary to the first region; and
one or more third regions complementary and hybridized to the first region, wherein cleavage of the one or more second regions results in dehybridization of the third region from the first region, resulting in an unblocked nucleic acid molecule.

15. The reaction mixture of claim 14, wherein a K_{d} of the blocked nucleic acid molecule binding to the RNP2 complex is at least 10¹⁰-fold greater than a K^{d} of the blocked nucleic acid molecule binding to the RNP2 when unblocked.

16. The composition of matter of claim 16, wherein a K^{d} of the blocked nucleic acid molecule binding to the RNP2 complex is at least 10⁵-fold greater than a K^{d} of the blocked nucleic acid molecule binding to the RNP2 when unblocked.

17. The reaction mixture of claim 13, further comprising reporter moieties.

18. The reaction mixture of claim 17, wherein the reporter moiety comprises a DNA, RNA or chimeric nucleic acid molecule.

19. The method of claim 17, wherein the reporter moiety comprises a fluorescent, chemiluminescent, radioactive, colorimetric or other optical signal.

20. A method of detecting a target nucleic acid of interest in a sample comprising the steps of:
providing the reaction mixture of claim 13;
contacting the reaction mixture with the sample under conditions that allow the target nucleic acid of interest in the sample to bind to RNP1; wherein upon binding of the target nucleic acid of interest RNP1 becomes active initiating trans-cleavage of at least one of the blocked nucleic acid molecules thereby producing at least one unblocked nucleic acid molecule, and wherein the at least one unblocked nucleic acid molecule binds to RNP2 initiating cleavage of at least one further linear blocked nucleic acid molecule; and
detecting the cleavage products, thereby detecting the target nucleic acid of interest in the sample.

21. The method of claim 20, further comprising reporter moieties.

22. The method of claim 20, wherein the RNP2 in the reaction mixture comprises the stabilization variant LbCas12a nuclease comprising SEQ ID NO: 2.

23. The method of claim 20, wherein the RNP2 in the reaction mixture comprises the stabilization variant LbCas12a nuclease comprising SEQ ID NO: 3.

24. The method of claim 20, wherein the RNP2 in the reaction mixture comprises the stabilization variant LbCas12a nuclease comprising SEQ ID NO: 4.

25. The method of claim 20, wherein the RNP2 in the reaction mixture comprises the stabilization variant LbCas12a nuclease comprising SEQ ID NO: 5.

26. The method of claim 20, wherein the RNP2 in the reaction mixture comprises the stabilization variant LbCas12a nuclease comprising SEQ ID NO: 6.

27. The method of claim 20, wherein the RNP2 in the reaction mixture comprises the stabilization variant LbCas12a nuclease comprising SEQ ID NO: 7.

28. The method of claim 20, wherein the RNP2 in the reaction mixture comprises the stabilization variant LbCas12a nuclease comprising SEQ ID NO: 8.

29. The method of claim 20, wherein the RNP2 in the reaction mixture comprises the stabilization variant LbCas12a nuclease comprising SEQ ID NO: 9.

30. The method of claim 20, wherein the RNP2 in the reaction mixture comprises the stabilization variant LbCas12a nuclease comprising SEQ ID NO: 10.


SEQUENCE LISTING (12 sequences)

SEQ ID NO: 1 - protein, 1228 aa, unidentified
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 2 - protein, 1228 aa, synthetic construct
MSKLEKFTNSYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 3 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRSINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 4 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKSLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 5 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDSHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 6 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKSPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 7 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKISELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 8 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 9 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVSLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 10 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLSEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 11 - protein, 1228 aa, synthetic construct; identical to SEQ ID NO: 9

SEQ ID NO: 12 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKSLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKSPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKISELVEKYDAVIALEDLNSGFKNSRVKVEKQVYQKFEKMLIDKLNYMVDKKSNPSATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVSLTSAYKELFNKYGINYQQGDIRALLSEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH